(19) United States
(12) Patent Application Publication
(10) Pub. No.: US 2005/0276766 A1
(43) Pub. Date: Dec. 15, 2005

(54) Methods and compositions for increasing skin remodeling

(75) Inventor: Loren R. Pickart, Bellevue, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Skin Biology, Inc., Bellevue, WA (US)

(21) Appl. No.: 10/956,450

(22) Filed: Oct. 1, 2004

Related U.S. Application Data
(60) Provisional application No. 60/507,755, filed on Oct. 1, 2003.

(51) Int. Cl. A61K 7/135; A61K 7/32

(52) U.S. Cl. 424/62

(57) ABSTRACT

The present invention provides compositions and methods for accelerating the remodeling of skin and restoring the properties of a younger skin. This includes removal of skin imperfections, signs of aging, and better moistization of skin in warm-blooded animals. The compositions useful in these methods, including pharmaceutical compositions, are prepared from combinations of copper peptides or copper amino acids and salicyclic acid.

### CROSS-REFERENCES TO RELATED APPLICATIONS

The present application claims benefit to U.S. Provisional Application No. 60/507,755, filed Oct. 1, 2003, the entire disclosure of which is incorporated herein by reference.

### BACKGROUND OF THE INVENTION

During human aging, skin becomes thinner and accumulates various blemishes, lesions and imperfections. The structural proteins are progressively damaged causing collagen and elastin to lose their resiliency. The skin's water-holding proteins and sugars diminish, the dermis and epidermis thin, the capillary network becomes disorganized, and the subcutaneous fat cells diminish in number. These effects are further intensified by decades of exposure to ultraviolet rays, irritants, allergens, and various environmental toxins. The result is a dry, wrinkled, inelastic skin populated by unsightly lesions.

Restoration to a biologically younger skin morphology requires two linked processes: (1) the removal of damaged proteins and aberrant skin lesions, and (2) their replacement with normal, blemish free skin. This process is similar to the remodeling phase of wound healing in which scar tissue is removed over several years to slowly restore the skin to its original state. In young children this process functions efficiently and skin damage is rapidly removed. But in adults this process slows drastically and various skin lesions may persist for years or decades.

The treatment of skin imperfections such as scars, solar keratosis (sun damage marks), age spots, skin tags, calluses, keloids, moles, pigmentations, stretch marks and other dermatological signs of aging remains a major health and cosmetic concern despite the development of numerous treatments such as the use of silicone sheets, scar subcision, deep chemical peels, laser resurfacing, dermabrasion and so forth. The problem with current techniques for removal of skin imperfections is that they all are poorly effective, expensive and often painful. If the dermatologist or esthetician performing the procedure is not highly skilled, then the results are often unsatisfactory and can produce further scarring.

Retinoic acid slowly remodels skin but at the price of chronic irritation and redness. Collagen inducing peptides, melatonin, and vitamin C increase skin collagen but skin also needs rebuilding of elastin, water-holding proteoglycans, and its microcirculation. Furthermore, collagen inducing peptides and proteins, such a TGF-beta 1, fibronectin binding peptides, and similar molecules, often produce unacceptable skin thickening and scarring. Controlled skin damage (i.e., lasers, peels, dermabrasion) works well only if there is a vigorous post-therapy regenerative response by the damaged skin.

An alternative method to remove skin imperfection is to enhance the natural biological processes that restore skin morphology to that more like a biologically younger skin by a process called “skin remodeling.” Evolution has designed the skin to heal wounds quickly to prevent blood loss and infection. Scars are manufactured from a rapidly formed “collagen glue” that the body deposits into an injured area for protection and strength. In ideal skin healing, the wounded skin is rapidly closed, then by the process of skin remodeling the healed area is slowly reconstructed to remove the residual scars and imperfections. This smooths the skin and blends the skin with nearby undamaged skin. Scar collagen is removed and replaced with a mixture of skin cells and invisible collagen fibers. This skin remodeling may continue in a skin area for 10 years. In children the remodeling rate is high and scars and other types of imperfections are usually rapidly removed from injured or disfigured skin areas. But as we reach adulthood, this rate diminishes and small scars and lesions may remain for years.

### SUMMARY OF THE INVENTION

The present invention provides compositions and methods for accelerating the remodeling of skin and restoring the properties of a younger skin. This includes removal of skin imperfections, signs of aging, and better moisturization of skin in warm-blooded animals. The compositions useful in these methods, including pharmaceutical compositions, are prepared from combinations of copper peptides or copper amino acids and salicylic acid.

Thus, the present invention provides therapeutic treatments to enhance the removal of skin imperfections by the use of a combination of salicylic acid and metal ions complexed with synthetic peptides or biological peptones, alone or in combination with other agents, to speed the removal of skin remodeling. These actions are obtained with compositions formulated at the normal pH ranges of human skin, that is between about 5.0 and about 7.0. Current effective hydroxy acid exfoliation systems must be used at very low pHs, often such as 2.8 for salicylic acid, to obtain significant actions. Such low pHs are irritating to skin and can burn the skin. The present invention achieves desired therapeutic and cosmeceutical actions on skin and skin renewal but with pH ranges that are gentle and substantially non-irritating to the skin.

The present compositions and methods typically employ formulations of salicylic acid but also extend to formulations of other salicylates, such as, for example, acetyl salicylic acid, salsalate, 2-hydroxy benzoic acid, homosolate, di(3-aminopyridine)disalicylate, 2.5-diisopropyl-salicylic acid. These salicylates are provided for purposes of illustration only, a wide variety of salicylates being available and suitable for incorporation into the present compositions and methods.

The methods to enhance skin remodeling comprise administering to the skin lesion a therapeutically effective amount, of a composition, which comprises the organic metal complex and a salicylate.

In further embodiments of the invention, the compositions and methods are used for reducing surgical scar formation by use of metal complexes and salicylate immediately after surgery. The methods comprise administering to the skin incision area a prophylactically effective amount of a composition, which comprises the organic metal complex and a salicylate.

### DESCRIPTION OF THE SPECIFIC EMBODIMENTS

By definition, both synthetic peptides and peptones obtained by the hydrolysis of larger peptides and proteins are referred to herein as “peptide”.

By definition, both metal complexes of synthetic peptides and metal complexes of peptones are referred to as “peptide-metal complexes” or, peptide-copper complexes, depending on the particular metal employed in the complex.

By definition, metal complexes of amino acids are referred to as “amino acid-copper complexes”.

Salicylic acid in the resultant solutions is referred to even though it is recognized that the molecule would typically exist, because of changes in ionization at various pHs, as a mixture of salicylic acid and ionic salicylate.

Peptides and Peptones

Synthetic peptides include molecules such as Gly-His-Lys, although virtually any small peptide appears to function similarly. Synthetic peptides are commercially available from a wide variety of sources such as Sigma Chemical Company, St. Louis, Mo.

Peptones are generally comprised of intermediate polypeptide products and mixtures of small peptides, formed in partial hydrolysis of proteins. Among the types of protein digests useful in the invention are digests of soybean protein, casein, collagen, elastin, meat products (erg., PRIMATONE), such as beef, liver, silk protein and so forth. By peptone digest is meant that the protein is degraded by enzymatic digestion or by acid or base hydrolysis, according to well known procedures, such as described in Remington's Pharmaceutical Sciences, Mack Publishing Co., Easton, Pa. pp. 428-429 (1975), which is incorporated herein by reference, using enzymes such as papain, etc. Alternatively, the digests may be produced by bacterial or yeast cultures that internally hydrolyze proteins and secrete the subsequent peptides, or by bacterial cultures that secrete hydrolytic enzymes in a culture medium which then hydrolyze proteins in the culture medium. Many peptone digests are widely available commercially, such as from Sigma Chemical Company, St. Louis, Mo.

Amino acids, such as L-tryptophan, are widely available commercially, such as from Sigma Chemical Company, St. Louis, Mo.

Metal Salts and Metal Complexes

Ionic metal complexes of copper, tin and zinc, or the salts thereof, such as sulfate, acetate, phosphate, gluconate, di-D-gluconate, acetyl tyrosinate, asparatate, methylsilanol, acetylmethionate, bis(N-acetyl-1-methioninato-, PCA (L-proline, 5-oxo-), PCA methysilanol, usnate (1,3-(2H, 9bh)-dibenzofurandione, 2,6-diacetyl-7,9-dihydroxy-8,9b-dimethyl-) and other complexing agents.

Such metal complexes are widely available commercially, such as from Sigma Chemical Company, St. Louis, Mo.

Salicylic Acid

Salicylic acid (99% pure) is obtained from Ruger Chemical Company, Irvington, N.J. Other salicylates are widely available from a variety of commercial sources.

Formation of Combinations of Copper Organic Complexes and Salicylic Acid

A peptide is combined with an amount of an aqueous solution of transition metal salt sufficient to form a complex. The pH of the solution is then raised to 6.0 and the salicylic acid is dissolved into the solution.

Typically the complex is then combined with a pharmaceutically acceptable carrier to form a cream, lotion, or solution in a concentration of from about 0.1% to about 25% peptide-metal complex or more. The preparation may be sterilized or pasteurized, as desired, without destroying the skin remodeling and lesion removal activity of the peptide-metal/salicylate complex.

### Description of the Specific Embodiments

To produce the complexes useful in the present invention, the peptides are complexed with one or more ionic transition metals, such as copper, tin, zinc, or the salts thereof, such as sulfate, acetate, phosphate, etc. In one method for preparing the organic-metal complex, the peptide is dissolved in warm water (about 40 C to 60 C) at a concentration of about 20 to 50% (weight/volume), then mixed with a aqueous solution of a metal salt complex at a salt concentration of about 10 to 50% (w/v).

By complexed is meant that the peptides and metal ions form electrostatic bonds, although this mechanism is offered by way of possible explanation only and not by way of limitation.

The pH of the mixture is adjusted (with sodium hydroxide or other acceptable agent) to a pH between 5.0 and 7.0. A quantity of about 5 to 25% salicylic acid is then added to the mixture and dissolved by stirring. After this procedure the pH, which tends to become lower, is again raised to between 5.0 to 7.0, as desired. Other aqueous components, as desired, are added, followed by blending in of carriers, smootheners, etc. for preparing a final formulation.

The peptide-metal/salicylic acid combinations of the invention may be administered for a variety of therapeutic, prophylactic or cosmetic uses to humans or in veterinary applications to other warm-blooded animals. Among veterinary animals particularly well suited for treatment with the present compositions are species of equine, bovine, porcine, ovine, caprine, canine, avian, feline, etc.

The compositions, pharmaceutical preparations, and cosmeceutical formlations of the present invention are intended for local, topical, oral, or subcutaneous injection administration for a prophylactic, therapeutic and/or cosmetic treatment regimen, to facilitate natural skin remodeling. Preferably, the compositions, including pharmaceutical compositions, are administered are administered locally, eg., topically, as a paste, cream, ointment, salve, lotion, gel, spray, aerosol, or the like.

For administration to warm-blooded animals, the peptide-metal/salicylate compositions can be sterilized and incorporated in pharmaceutical or veterinary formulations. Such compositions can be sterilized by conventional, well-known sterilization techniques, eg., boiling or pasteurization, without substantially adversely affecting the biological activity of the peptide-metal/salicylic aid complexes. The compositions may contain pharmaceutically acceptable auxiliary substances as required to approximate physiological conditions and as necessary to prepare compositions for convenient administration, such an pH adjusting and buffering agents, and delivery vehicles.

Actual methods for preparing pharmaceutically administrable compounds will be known or apparent to those skilled in the art and are described in detail in, for example, Remington's Pharmaceutical Science, supra.

Depending on the intended mode of administration and the intended use, the compositions may be in the form of solid, semi-solid, or liquid dosage forms, such, for example, as powders, granules, crystals, liquids, suspensions, liposomes, pastes, creams, salves, etc., and may be in unit-dosage forms suitable for administration of relatively precise dosages. The compositions may include a conventional pharmaceutical carrier or excipient and, in addition, may include other medicinal agents, growth factors, wound sealants, carriers, etc., as further described below.

For semi-solid compositions, as would be appropriate for pastes and creams intended for topical administration, the peptide-metal complexes can be provided separately kwith salicylatesor may be compounded with conventional nontoxic carriers such as, for example, aloe vera gel, squalane, glycerol stearate, polyethylene glycol, cetyl alcohol, stearic acid, and propylene glycol, among others. Such compositions may contain about 1-50% active ingredient of either copper-peptides or salicylic acid, more preferably about 1-25%.

Thus, the final concentration of copper or other metal in a formulation can range from about 0.1 or 0.15% (w/v) up to 0.4 to 0.8% or 1.6%, and in some instances up to 2 to 5% or more, although it will typically be desirable to employ the lowest final concentration of copper as possible which achieves the desired effect.

The final concentration of salicylic acid in a formulation can range from about 0.5 up to 40% (w/v) although it will typically be desirable to employ the lowest final concentration of salicylic acid as possible which achieves the desired effect.

The concentration of the peptide-metausalicylic acid combinations in these formulations can vary widely, and will be selected primarily by intended use, viscosities, etc., in accordance with the those skilled in this art; for example, see Remington's Pharmaceutical Science, 17th ed., Mack Publishing Company, Easton, Pa. (1985), which in incorporated herein by reference. The composition or formulation to be administered will, in any event, contain a quantity of the peptide-metal/salicylate or peptone-metal/salicylate complexes sufficient to achieve the desired therapeutic, prophylactic or cosmetic effect in the subject being treated.

The compositions of the invention are administered to a warm-blooded animal, such as humans, already suffering from a skin lesions or prophylactically after surgical incision to reduce scar formation. Amounts adequate to accomplish these effects are defined as a “therapeutically effective doses.” Amounts effective for this use will depend on the severity of the lesion or surgical but generally range from about 0.1 mg to about 50 mg per day of peptide-metal complex per day per square centimeter of wound site, with dosages of from about 10 mg to about 25 mg per day per square centimeter of wound site being more commonly used. Maintenance dosages over a prolonged period of time may be adjusted as necessary.

For veterinary uses higher levels may be administered as necessary. Determining actual amounts of the peptide-metal/salicylate combinations necessary to treat a particular condition as described herein will be through standard empirical methods well known in the art.

In prophylactic applications, compositions containing the peptide-metal/salicylate complexes are administered to a host susceptible to or otherwise at risk of skin lesions or similar damage, to enhance the host's own skin remodeling. Such an amount is defined to be a “prophylactically effective dose.” In this use, the precise amounts again depend on the host's condition and general state of health, but generally range from about 0.1 mg to about 10 mg per day per square centimeter of skin, more commonly from about 1 mg to about 3 mg per cm2 of skin per day. Single or multiple administrations of the compositions can be carried out.

The peptide-metal/salicylic acid complexes of the invention may be administered in relatively large amounts without serious side effects, although indiscriminate use may produce discoloration of the skin.

### EXAMPLES

The following examples are offered by way of illustration, not by way of limitation.

### Example I

Preparation of Active Peptide-Metal/Salicylic Acid Combinations

This Example describes methods used in the preparation of peptide-metal and salicylic acid combinations having biological activities described further below. Soybean peptone was obtained from Sigma Chemical Company, St. Louis, Mo. (type IV, number P 0521), as was cupric chloride hydrate (no. C 6641) obtained from Aldrich Chemical Company, Milwaukee, Wis. Salicylic acid (99% pure) was obtained from Ruger Chemical Company, Irvington, N.J.

Soybean peptones (enzymatic digests of soybean protein) were dissolved in warm water (40 C) at a concentration of 20% (weight/volume), then mixed with an aqueous solution of a metal salt (copper(II) chloride) at a salt concentration of 20% (w/v). The pH of the mixture was raised to 6.5, then a sufficient quantity of salicylic acid was added to give final concentration of salicylic acid in the solution of 0.5% to 3.0% as desired.

Other types of enzymatic protein digests such as those of casein, collagen, elastin, meat products, silk protein and the like, and other metal salts of the metals, such as sulfate, acetate, phosphate and so forth work similarly.

The synthetic peptide Gly-His-Lys was purchased from commercial sources although other any small peptides function similarly. Synthetic peptides are commercially available from sources such as Sigma Chemical Company, St. Louis, Mo. The peptides were complexed to metals as described above. (See also, Pickart and Thaler, Nature New Biol. 1973, 243, pp. 85-7).

Peptides are dissolved in warm water (40 C) at a concentration of 20% (weight/volume), then mixed with a aqueous solution of a metal salt (copper(II) chloride) at a salt concentration of 20% (w/v).

A cream base for testing was prepared by blending sufficient copper chloride for a final concentration of 0.6% copper ion, sufficient soy peptone (or synthetic peptides) for a final concentration of 4% dry weight, and sufficient salicylic acid for a final concentration of 2% salicylic acid into an application cream for application to the skin comprising, in approximate amounts (final concentrations, w/v %): water, 60.08; cetyl alcohol, 5.0; stearic acid, 5.0; ARLACEL 165, 7.0; LEXOL EHP, 4.0; squalene, 5.0; copper chloride-2H₂₀, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01. The pH of the cream was 6.5, but creams from pH 5.0 to 7.0 gave essentially similar results.

### Example II

Reduction in Scar Formation after Surgical Incisions with Peptide Copper Complex and Peptide-Copper or Amino Acid-Copper Complexes and Salicylic Acid Combinations

This Example describes the use of a pastes prepared with the complexes of various copper peptides or copper complexes of amino acids and combinations of copper peptides or copper complexes of amino acids and salicylic acid to reduce scarring after surgery in animals.

Surgical incisions (1.25 cm) were made on the backs of anesthetized, 35 gram, Swiss-Webster mice. To allow normal scar development, there was no treatment after surgery. But 24 hours after surgery, the scars were covered with a cream containing either copper peptide alone or in combination with salicylic acid. Control wounds were untreated. As seen in Table 1, wounds treated with the copper peptide/salicylic acid combination had much less post-surgical scar formation than control wounds or those treated with only copper peptide containing cream.

For comparison, the amount of scar formation was rated from 0 (little or no scarring) to 4 (maximum observed scarring) after seven days.

Table 1: Effect of cream at pH 6.5 with either peptide-copper complex or amino acid-copper complex or the combination of peptide copper/salicylic acid combination or amino acid copper/salicylic acid combination on scar formation

| Test group | Scar Formation score |
|---|---|
| (15 mice) | after 7 days |
| Control | 3.8 ± 0.7 |
| Soy Peptone-Copper(II) | 1.6 ± 0.3 |
| complex | |
| Soy Peptone-Copper(II)/ | 0.7 ± 0.2 |
| Salicylic acid combination | |
| Gly-His-Lys-Copper(II) | 2.7 ± 0.6 |
| Gly-His-Lys-Copper(II)/ | 1.3 ± 0.5 |
| Salicylic acid combination | |
| L-tryptophane-Copper (II) | 3.7 + 0.6 |
| L-tryptophane-Copper (II) | 3.2 + 0.2 |
| Salicylic acid combination | |

### Example III

Removal of Multiple Facial Keratosis with Copper Peptone/Salicylic Acid Combination in a Cream Base

This Example demonstrates the removal of facial keratosis with the the cream containing (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A 33 year old woman with heavy facial sun damage, keratosis, and fine lines reported that use of the cream removed the lesions within one month.

### Example IV

Removal of Skin Damage Around the Eyes with Peptone-Copper/Salicylic Acid Combination in a Cream Base

This Example demonstrates the removal of skin damage around the eyes with the cream base containing (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A 30 year old professional model used the cream with copper peptide/salicylic acid combination around her eyes and observed a rapid clearing of sun damage and disfiguring fine lines.

### Example V

Removal of Acne scars with Peptone-Copper/Salicylic Acid Combination in a Cream Base Removal of Acne Scars with

This Example demonstrates the removal of acne scars with the cream base containing (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A 27 year old women with pitted acne reported that daily application of the cream with peptide copper/salicylic acid combination removed 90% of her scars in two months.

### Example VI

Removal of Pitted Acne Scars with Peptone-Copper/Salicylic Acid Combination in a Cream Base

This Example demonstrates the removal of pitted acne scars with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A 34 year old woman had severe pitted acne scarring. Her esthetician used subcision (needling) to break up the pitted scar tissue. Then the cream with peptide copper/salicylic acid combination was used to speed healing. This procedure was reported to remove the pitted scars within a month.

### Example VII

Removal of Reddish Acne Scars with Peptone-Copper/Salicylic Acid Combination in a Cream Base

This Example demonstrates the removal of reddish acne scars with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A 21 year old male used the cream with peptide copper/salicylic acid combination for five weeks and reported the clearing of reddish acne scars.

### Example VIII

Removal of Depressed Acne Scars with Peptone-Copper/Salicylic Acid Combination in a Cream Base

This Example demonstrates the removal of depressed acne scars with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A 47 year old male had severe adult acne in his 20's that left uneven skin with depressed areas. Numerous painful dermabrasion sessions had only a minor clearing of the scars. He reported that one month of using the cream with peptide copper/salicylic acid combination produced a marked reduction in the scars and a smoother and healthier looking face.

### Example IX

Removal of Cystic Acne Scars with Peptone-Copper/Salicylic Acid Combination in a Cream Base

This Example demonstrates the removal of cystic acne scars with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A 23 year old women had chronic problems with cystic acne and resultant scars. She applied the cream with peptide copper/salicylic acid combination nightly to any acne breakout and reported that the cysts heal to a flatten, reddish mark that eventually became normal colored skin.

### Example X

Removal of Skin Tags with Peptone-Copper/Salicylic Acid Combination in a Cream Base

This Example demonstrates the removal of skin tags with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A women applied the cream containing copper peptide/salicylic acid combination to skin tags and reported that within a month they had shrunk and fallen off.

### Example XI

Removal of Facial Moles with Peptone-Copper/Salicylic Acid Combination in a Cream Base

This Example demonstrates the removal of facial moles with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A woman applied the cream with peptide copper/salicylic acid combination to several facial moles and observed their disappearance within two months.

### Example XII

Removal of Sun Damage Marks with Peptone-Copper/Salicylic Acid Combination in a Cream Base

This Example demonstrates the removal of sun damage marks with the cream base prepared with final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A 52 year old woman applied the cream with peptide copper/salicylic acid combination on her upper chests and breasts for two months and reported that sun damage marks were removed.

### Example XIII

Removal of Age Spots with Peptone-Copper/Salicylic Acid Combination in a Cream Base

This Example demonstrates the removal of age spots with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A 72 year old man used the cream with peptide copper/salicylic acid combination for six weeks and removed age spots from the skin on his head and face.

### Example XIV

Removal of Facial Wrinkles with Peptone-Copper/Salicylic Acid Combination in a Cream Base

This Example demonstrates the removal of facial wrinkles with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A 75 year old women used the cream with peptide copper/salicylic acid combination and reported that her facial wrinkles were much less pronounced.

### Example XV

Removal of Skin Damage Around the Eyes with Peptone-Copper/Salicylic Acid Combination in a Cream Base

This Example demonstrates the removal of skin damage around the eyes with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A 40 year old model used the cream with peptide copper/salicylic acid combination around her eyes for two months and observed the removal of fine lines and a better tone and color in the skin.

### Example XVI

Removal of Keloid Scars with Peptone-Copper/Salicylic Acid Combination in a Cream Base

This Example demonstrates the removal of keloid scars with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A 21 year old man used the cream with peptide copper/salicylic acid combination for three months on raised keloid acne scars on his back and observed their disappearance.

### Example XVII

Removal of Stretch Marks with Peptone-Copper/Salicylic Acid Combination in a Cream Base

This Example demonstrates that the removal of stretch marks with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A woman age 49 had stretch marks from three pregnancies. She applied the cream for four months and found they had vanished.

### Example XVIII

Removal of Burns Scars with Peptone-Copper/Salicylic Acid Combination in a Cream Base

This Example demonstrates the removal of pitted acne scars with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A women age 21 had two-month old burn scars on her hand. The use of the cream totally restored normal skin.

### Example XIX

Removal of Surgical Scars with Peptone-Copper/Salicylic Acid Combination in a Cream Base

This Example demonstrates the removal of surgical scars with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A man age 65 had 12-year-old surgical scars on leg. After using the cream for four months, the scars had vanished.

### Example XX

Removal of Foot Calluses with Peptone-Copper/Salicylic Acid Combination in a Cream Base

This Example demonstrates the removal of foot calluses with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A women of age 59 had chronic problems with foot calluses. After using the cream for three months, the calluses had vanished.

### Example XXI

Removal of Foot Calluses with Peptone-Copper/Salicylic Acid Combination to Improve Skin Moisturization

This Example demonstrates the removal of foot calluses with the cream base prepared with (final concentrations, w/v %): water, 60.08; cetyl alcohol, 5.0; stearic acid, 5.0; ARLACEL 165, 7.0; LEXOL EHP, 4.0; squalene, 5.0; copper chloride-2H20, 1.6; soybean peptone, 6.0; mineral oil, 3.0; salicylic acid 2.0, allantoin 0.5; GERMABEN-II-E, 1.0; herbal fragrance, 0.1; aloe vera powder, 0.1; vitamin A-D, 0.01; vitamin E, 0.01.

A women of age 74 had chronic problems with extremely dry and cracking skin. After using the cream for one months, her skin was well moisturized and did not develop cracks.

All publications and patents mentioned in this specification are indicative of the level of skill of those skilled in the art to which this invention pertains. All publications and patents are herein incorporated by reference to the same extent as if each individual publication or patent was specifically and individually indicated to be incorporated herein by reference.

Although the foregoing invention has been described in some detail by way of illustration and example for purposes of clarity of understanding, it will be obvious that certain changes and modifications may be practiced within the scope of the appended claims.

1. A method for stimulating the remodeling of a skin blemish in a warm-blooded animal comprising administering to the skin blemish a composition that comprises an ionic metal-peptide complex and a salicylate in an amount effective to diminish or remove the skin blemish.

2. The method according to claim 1, wherein the skin blemish is a skin tag, acne scar, skin callus, benign skin mole, stretch mark, solar keratosis, keloid scar, burn scar, thickened sunspot, or surgical scar.

3. The method of claim 1, wherein the ionic metal is selected from the group consisting of copper(II), tin(II), tin(IV), and zinc(II), and therapeutically acceptable salts and complexes thereof.

4. The method of claim 3, wherein the ionic metal is copper(II) and therapeutically acceptable salts and complexes thereof.

5. The method according to claim 1, wherein the peptide of the ionic metal-peptide complex is an enzymatic hydrolysis of casein, collagen, elastin, meat products, silk protein, or soybean protein.

6. The method according to claim 1, wherein the peptide of the ionic metal-peptide complex is an acid hydrolysis of casein, collagen, elastin, meat products, silk protein, or soybean protein.

7. The method according to claim 1, wherein the peptide of the ionic metal-peptide complex is a basic hydrolysis of casein, collagen, elastin, meat products, silk protein, or soybean protein.

8. The method according to claim 1, wherein the peptide of the ionic metal-peptide complex is a bacterial hydrolysis of casein, collagen, elastin, meat products, silk protein, or soybean protein.

9. The method according to claim 1, wherein the peptide of the ionic metal-peptide complex is a chemically-synthesized copper binding peptide.

10. The method according to claim 1, wherein the composition is administered topically or by injection into the skin.

11. The method according to claim 1, wherein the ionic metal-peptide complex is combined with the salicylate and a carrier to form a cream or lotion.

12. The method according to claim 1, wherein the concentration of the ionic metal-peptide complex in the composition is 1% to 25%.

13. A method for inhibiting the development of scar development in a warm-blooded animal comprising administering to the site of tissue injury skin a composition that comprises an ionic metal-peptide complex and a salicylate in an amount effective to inhibit the development of scar tissue.

14. The method of claim 13, wherein the tissue is skin and the injury is an incision.

15. A method for improving moisturization of skin of a warm-blooded animal, comprising administering to the skin of said animal a composition that comprises an ionic metal-peptide complex and a salicylate in an amount effective to improve the moisturization of said skin.

16. A dermatological composition for administration to the skin of a mammal, which comprises an ionic metal-peptide complex, a salicylate, and a dermatologically acceptable carrier.

17. The dermatological composition of claim 16, wherein the ionic metal is copper(II) and therapeutically acceptable salts and complexes thereof.

18. The dermatological composition of claim 16, wherein the salicylate is selected from the group consisting of salicylic acid, acetyl salicylic acid, salsalate, 2-hydroxy benzoic acid, homosolate, di(3-aminopyridine)disalicylate, and 2.5-diisopropyl-salicylic acid.

19. The dermatological composition of claim 16, wherein the salicylate is salicylic acid.
